(19) United States
(12) Patent Application Publication
(10) Pub. No.: US 2014/0346073 A1
(43) Pub. Date: Nov. 27, 2014

(54) Stable Multi-Purpose Wound Dressing

(71) Applicants: Evelyn S. Sawyer, Freeport, ME (US); Paul A. Janmey, Media, PA (US)

(72) Inventors: Evelyn S. Sawyer, Freeport, ME (US); Paul A. Janmey, Media, PA (US)

(73) Assignee: SEA RUN HOLDINGS, INC., Freeport, ME (US)

(21) Appl. No.: 14/325,750

(22) Filed: Jul. 8, 2014

Related U.S. Application Data
(63) Continuation-in-part of application No. 13/208,479, filed on Aug. 12, 2011, now Pat. No. 8,771,684.

(51) Int. Cl. A61L 15/38; A61F 13/00; B65B 31/04; A61L 15/44

(52) U.S. Cl. CPC A61L 15/38; A61L 15/44; A61F 13/00072; B65B 31/04
USPC 206/440; 424/445; 424/94.64; 53/405; 427/2.31

(57) ABSTRACT

A wound dressing includes a backing material, and thrombin, applied to the backing material. Preferably, the backing material includes biodegradable polyester, such as electrospun polycaprolactone. The thrombin preferably includes salmon thrombin adsorbed onto the backing material. A process of fabricating a wound dressing includes applying thrombin to the backing material. Preferably, the thrombin is prepared from the plasma of a salmonid. The thrombin is applied to the backing material by adsorbing the thrombin onto the backing material, preferably after applying a detergent solution to the backing material. The dressing can be lyophilized and vacuum-sealed within an air-tight wrapping.

### CROSS-REFERENCE TO RELATED APPLICATIONS

This is a continuation-in-part of co-pending U.S. patent application Ser. No. 13/208,479, which was filed on Aug. 12, 2011, which in turn is a continuation-in-part of co-pending U.S. patent application Ser. No. 12/582,040, which was filed on Oct. 20, 2009. Claim is also made of the benefit of the filing date of U.S. Provisional Patent Application No. 61/393,678, filed on Oct. 15, 2010, pursuant to 35 U.S.C. ∪119(e).

### FIELD OF THE INVENTION

The present invention relates to dressings that treat the pain, bleeding, and inflammation that result from wounds incurred primarily in the field (for example, pre-hospital, combat, back-country), but can also be used for trauma or surgical wounds in-hospital.

### BACKGROUND OF THE INVENTION

U.S. Pat. No. 8,771,684 demonstrates alleviation of pain after tissue damage when followed by application of salmon thrombin to the injury site. The tissue damage results in nociceptive pain in the peripheral nervous system which signals the spinal cord and brain. Nociceptive pain includes the well-documented stimulation of peripheral pain fibers such as A- and C-fibers after surgery or other injury to skin, fascia, muscle, and bone.

Thrombin can act on mammalian cells through protease activated receptors (PARs) and other transmembrane proteins. The effect of these thrombin receptors on pain is complex, with some receptors on some cells inhibiting pain, and others promoting hyperalgesia (Garcia et al. 2010). In clinical practice however, mammalian thrombin is either ineffective for pain, or contraindicated as in cases of CNS injury. Therefore, the use of thrombin from any source as a treatment for pain is novel and represents a departure from current practice, which teaches against its use.

Thrombin polymerizes fibrinogen to form a clot (fibrin) and is generally recognized as a hemostatic agent. Previous studies have shown that salmon thrombin and human thrombin are interchangeable for fibrin formation. Michaud et al. 2002, emphasize the similarities of human and salmon thrombins in their function as hemostatic agents. Comparison of these two thrombins showed similar primary structure and specific enzyme activity with respect to activation of fibrinogen, and therefore salmon thrombin performs well as a hemostatic agent (Rothwell et al. 2005). Hemostatic agents are indicated for many surgical procedures and injuries, and there is a wide selection available (Spotnitz et al. 2008), but none address the accompanying pain.

Although bovine, human, and recombinant human thrombin are frequently used to control bleeding, these thrombins are pro-inflammatory in the CNS (Suo et al. 2004) and can in some cases exacerbate neuronal damage and pain (Wu et al. 2008), An ideal hemostatic/analgesic wound dressing should also be anti-inflammatory, and therefore the use of bovine, human, or recombinant human thrombin is not suitable. Evidence of the efficacy of salmon thrombin to reduce inflammation has only recently come to light, as presented by Weisshaar et al. 2011, and Smith et al. 2013.

The pain of combat wounds, surgery, and other tissue injury is most often treated with repeated injections of local anesthetics such as Marcaine® (bupivacaine), which can result in cardiovascular system toxicity (Mather 2010), or ketamine, which, like opioids, is effective for moderate to severe pain. However, these drugs often lead to a host of problems, including serious side-effects, abuse, and addiction.

Pusateri et al. 2006, with a focus on combat needs, listed the qualities required for a bandage or dressing to be suitable for field use. These included hemostatic, biodegradable, stabile, ready-to-use, simple to apply, lightweight, durable, and safe. Safety is not a concern with salmon thrombin because it contains no mammalian viruses or prions. Also, it does not produce antibodies that cross-react with host proteins or have any other adverse in vivo effects, as demonstrated in multiple animal models.

The present invention extends U.S. Pat. No. 8,771,684 by applying salmon thrombin to a backing that is preferably biodegradable, and stabilizing the resulting wound dressing for field or pre-hospital use. This novel dressing fulfills the criteria of Pusateri et al. and adds the important advantages of analgesia, and anti-inflammatory properties. There are well over one hundred patents on wound dressings and many hundred related publications (partial summary by MacPhee et al. 2004), but no single therapeutic dressing provides all the properties of the present invention.

### BRIEF SUMMARY OF THE INVENTION

According to an aspect of the invention, a wound dressing includes a backing material, and thrombin, applied to the backing material. Preferably, the backing material is at least partially biodegradable. For example, the backing material can include biodegradable polyester, such as electros pun polycaprolactone. Alternatively, the backing material can include other material, such as glycolic acid-based polymers, lactic acid-based polymers, glycolic acid-based copolymers, and/or lactic acid-based copolymers.

The thrombin preferably includes salmon thrombin. The wound dressing can also include fibrinogen, collagen, alginates, synthetic polymers, antibacterial factors, and/or antiviral factors, applied to the backing material or added to the thrombin.

The thrombin is adsorbed onto the backing material.

The wound dressing is in a lyophilized state.

The wound dressing can also include a detergent solution residue on the backing material.

According to another aspect of the invention, a wound dressing product includes an airtight wrapping in which the described wound dressing is enclosed.

Thus, alleviating pain, bleeding, and inflammation associated with tissue damage can be achieved by applying a stable, multi-purpose wound dressing that includes salmon thrombin lyophilized within an electrospun polycaprolactone (PCL) backing at a tissue damage site. Other substances may be added to the dressing such as fibrinogen, collagen, alginates, synthetic polymers, or antibacterial or antiviral factors, as long as they are in a dry form. Salmon thrombin can be the only thrombin included on the dressing, or other thrombin may be added. Other biodegradable materials such as glycolic acid- or lactic acid-based polymers or copolymers can be used for backing.

According to another aspect of the invention, a process of fabricating a wound dressing includes applying thrombin to a backing material. Preferably, the thrombin is prepared from the plasma of a salmonid. The thrombin is applied to the backing material by adsorbing the thrombin onto the backing material.

The process can also include applying a detergent solution to the backing material prior to adsorbing the thrombin onto the backing material. The detergent solution can be, for example, 0.5 mg/ml Tween 80.

The process can also include lyophilizing the backing material and adsorbed thrombin. Also, fibrinogen, collagen, alginates, synthetic polymers, antibacterial factors, and/or antiviral factors can be added to the thrombin and/or the backing material.

The backing material can be a biodegradable polyester.

According to another aspect of the invention, a process of manufacturing a wound dressing product includes vacuum-sealing the wound dressing described above within an air-tight wrapping.

Fabrication of the dressing begins with preparing the thrombin from the plasma of salmonids that are progeny of domesticated broodstock reared under consistent and reproducible conditions. Blood is obtained from the fish, plasma is separated from the blood, and the salmon prothrombin is extracted from the plasma. Preferably, the salmonid from which the blood is obtained is sexually immature, in the log-phase of growth, larger than two kilograms, and/or reared by standard husbandry methods. The blood can be obtained from the salmonid by rendering the salmonid to a level of loss of reflex activity and drawing blood from a caudal blood vessel. Prior to rendering the salmonid to a level of loss of reflex activity, the levels of proteolytic enzymes and non-protein nitrogen present in the blood of the salmonid can be reduced. The plasma can be separated from the blood by centrifuging the blood. Extracting the salmon thrombin from the plasma can include performing an extraction process on the plasma such that all process temperatures are no greater than 6° C., no cytotoxic chemical residues remain in one or more plasma-components, and no oxidation of plasma lipids occurs. An antioxidant and/or a protease-inhibitor can be added to the plasma prior to extracting the salmon thrombin. Preferably, the salmonid is an Atlantic salmon.

Thrombin can be purified from either fresh or frozen plasma by prothrombin precipitations and chromatographic techniques.

Alternatively, the salmon thrombin can be obtained by other fractionations.

Alternately, the salmon thrombin can be obtained by recombinant technology.

The thrombin is then adsorbed in an electrospun PCL backing, and the entire dressing is lyophilized. The thrombin/PCL dressing is then packaged, for example, under vacuum in an air-tight wrapping, such as a foil wrapping.

### BRIEF DESCRIPTION OF THE DRAWINGS

FIG. 1 is a photograph of the thrombin/PCL dressing as packaged and labeled.

FIG. 2 is a graph of the retained thrombin activity of two different dressings after storage at refrigeration (4-6° C.) and room temperature for 90 and 180 days.

FIG. 3 is a graph showing reduction of swelling (a marker of inflammation) in a rat paw after injury and treatment with salmon thrombin.

### DETAILED DESCRIPTION OF THE INVENTION

The process preferably begins with the consistent and reproducible conditions under which donor fish are reared. All fish used as plasma sources preferably are progeny of domesticated broodstock, inspected for fish disease according to the American Fisheries Society “Blue Book” standards, sexually immature, in the log-phase of growth, larger than two kilograms, reared by standard husbandry methods, and fed a commercially pelleted food appropriate to the species.

Water temperature at the time of bleeding is preferably 4° C. to 15° C. The fish are preferably starved for five days before bleeding to reduce proteolytic enzymes and non-protein nitrogen. Each fish is stunned by a blow to the head, or by immersion in ice-water, or in water containing CO₂ or other fish anesthetic, in order to stun the fish to a level of loss of reflex activity (unconsciousness). Whole blood is then drawn from the caudal artery or vein with a sterile needle and syringe or vacuum tube containing an anticoagulant such as ACD (acid citrate dextrose), trisodium citrate, or other anticoagulant commonly used in human blood-banking.

Whole blood is held, preferably for no more than four hours at 2-4° C., and then centrifuged at 2-4° C. Thrombin then can be prepared by the method of Michaud et al. 2002.

Briefly, prothrombin is extracted from plasma, for example, with a series of barium chloride and ammonium sulfate precipitations. The prothrombin is activated by Echis carinatus venom or equivalent proteolytic enzymes that cleave the site required to activate prothrombin, and the thrombin is passed over a heparin column or other binding matrix such as SP-Sepharose. Thrombin is eluted from the column with a high salt buffer and frozen at <−60° C. The thrombin may then be lyophilized. Alternately, plasma fractionation or recombinant techniques can be used. These techniques are illustrative of those currently in use, but other techniques for preparation of thrombin or its precursor prothrombin may be equally effective.

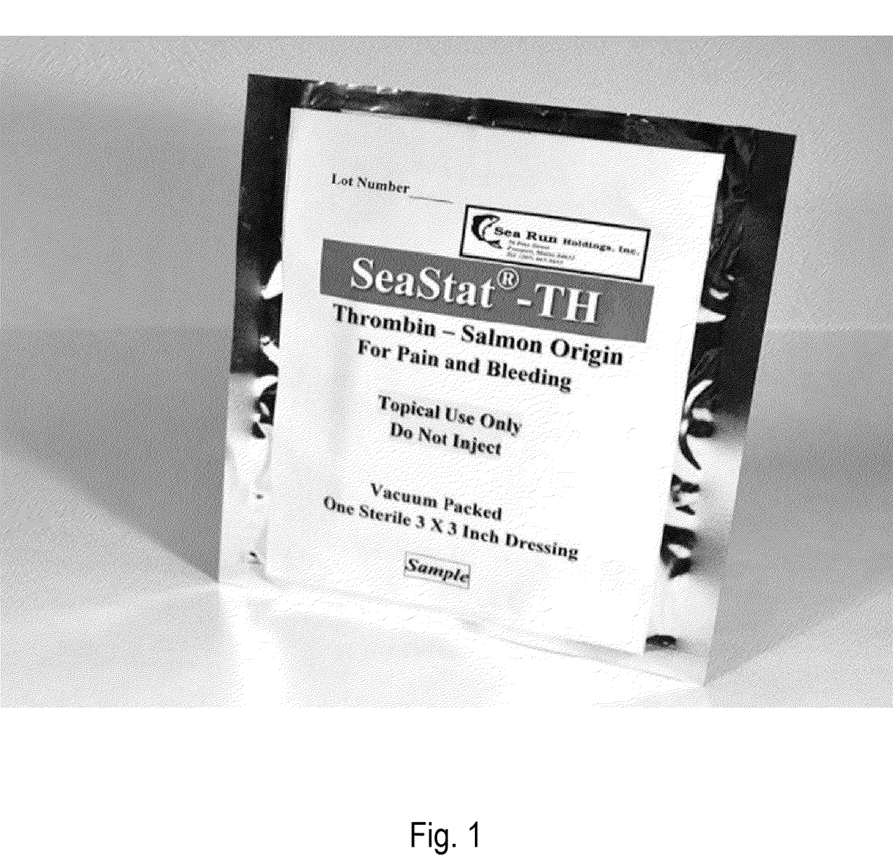

For example, frozen salmon thrombin was thawed at 4-6° C., analyzed for activity, and held on ice for less than one hour. Electrospun polycaprolactone (PCL), purchased from SNS Nanofiber Technology, was cut into 2 cm squares, and 200 μl of thrombin was applied to each square. Of course, PCL or other biodegradable polyester obtained from another source can be used. PCL is known to be hydrophobic, so it would not absorb the liquid thrombin. The PCL squares were therefore soaked in a detergent solution (0.5 mg/ml Tween 80) and the result was that the thrombin penetrated between the fibers and was adsorbed on the PCL, Other detergent solutions, such as Triton X-100, may be used in place of the Tween 80. Also, instead of soaking, the detergent solution can be applied by pipette, spraying, or any other method that allows it to soak into the backing material. Alternatively, the detergent solution can be mixed with the thrombin and applied to the backing material with the thrombin. The PCL squares (dressings) with the adsorbed thrombin were then frozen at −80° C., lyophilized, vacuum packed in foil, and stored at refrigerator or room temperature (FIG. 1).

Stability

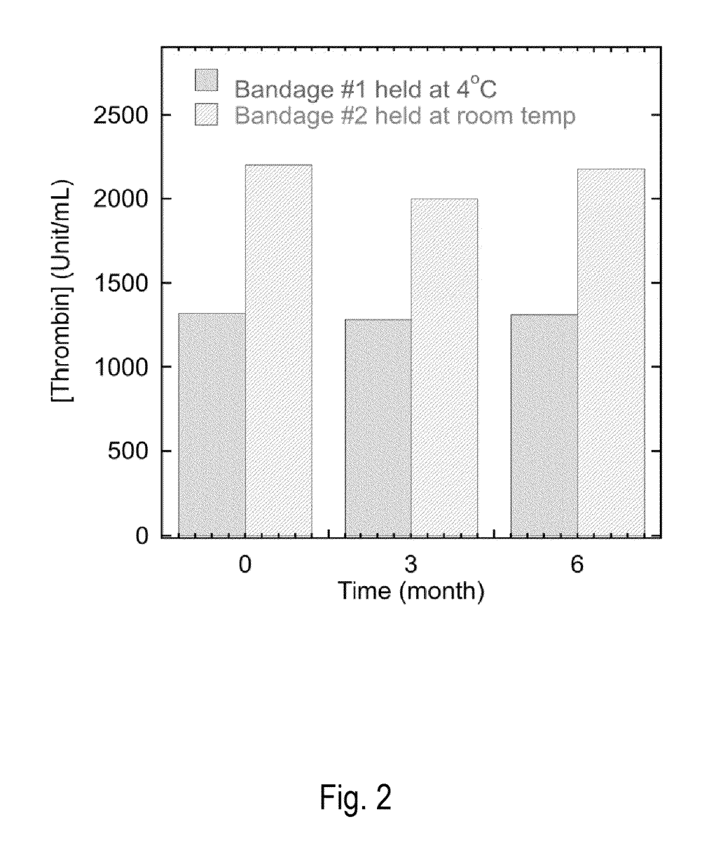

After 90 and 180 days, the dressings were rehydrated with 200 μl of distilled water and held at room temperature for less than 5 minutes. The eluate containing thrombin was then pressed from the dressing and analyzed for thrombin activity. FIG. 2 shows that more than 90% of the original activity was retained for at least 180 days, demonstrating the stability of the dressing.

Analgesia

Although mammalian thrombins are contraindicated for treatment of pain, we have found that salmon-derived thrombin applied to an injury site is highly effective for reducing pain originating from injury to nerves and other tissue. This effectiveness is independent of any healing or structural relief provided at the injury site; that is, the thrombin provides analgesic relief. The beneficial effects of salmon thrombin are likely related to differing cellular receptors. In contrast to the very similar reactivity of salmon and human thrombin to fibrinogen-based substrates (Michaud et al. 2002), we have found substantial differences in their ability to activate protease activated receptors (PAR). At 37° C. salmon thrombin cleaves PAR1 approximately three times more slowly than human thrombin (p=0.0013). These findings illustrate some of the functional differences in mammalian and salmon thrombin.

Hemostasis

Salmon thrombin is an effective hemostat (Rothwell et al., 2005), and the pain of serious injury is frequently accompanied by bleeding. Therefore, a dressing that would treat both pain and bleeding is desirable.

Anti-Inflammation

We have demonstrated the anti-inflammatory properties of salmon thrombin by experiments in a rat injury mode as shown in the following example. This anti-inflammatory effect adds a new and important dimension to the hemostatic/analgesic dressing.

### Example 1

As an example of inflammation after injury, we chose a rat incisional model of post-surgical swelling and inflammation. The rat plantar-incision model is a well-accepted and predictive model of human swelling and inflammation. It allows investigation of inflammation by comparing the effects of test substances during and after surgery (Whiteside et al. 2004; Brennan et al. 1996). Rats were anesthetized with isoflurane (2-3%) vaporized in a nose-cone. The plantar left hind paw was prepared in a sterile manner with an iodine solution and 70% ethanol. A 1 cm-long incision starting 0.5 cm from the heel and extending toward the toes was made with a number 10 blade, through the skin and fascia of the plantar aspect of the paw including the underlying muscle (Brennan et al. 1996). The plantaris muscle was then elevated and longitudinally incised, leaving the muscle origin and insertion intact. The wound was blotted with a gauze pad and 50 μl salmon thrombin or a control (distilled water) was applied directly to the wound cavity. The skin was then closed with two mattress sutures of 5-0 nylon. At the end of surgery, anesthesia was stopped, and rats were allowed to recover before being returned to their cages.

Before surgery and 12, 24, and 48 hours after surgery, groups of rats (N=−4) were evaluated for inflammation by measuring paw volume using an Ugo-Basile paw plethysometer. The rat paw is placed initially in a calibrated cylinder filled with a mixture of distilled water, salt and a wetting agent. Baseline and post-surgery paw volumes were obtained by measurement of the solution displacement, with the value being an average of two or three separate determinations.

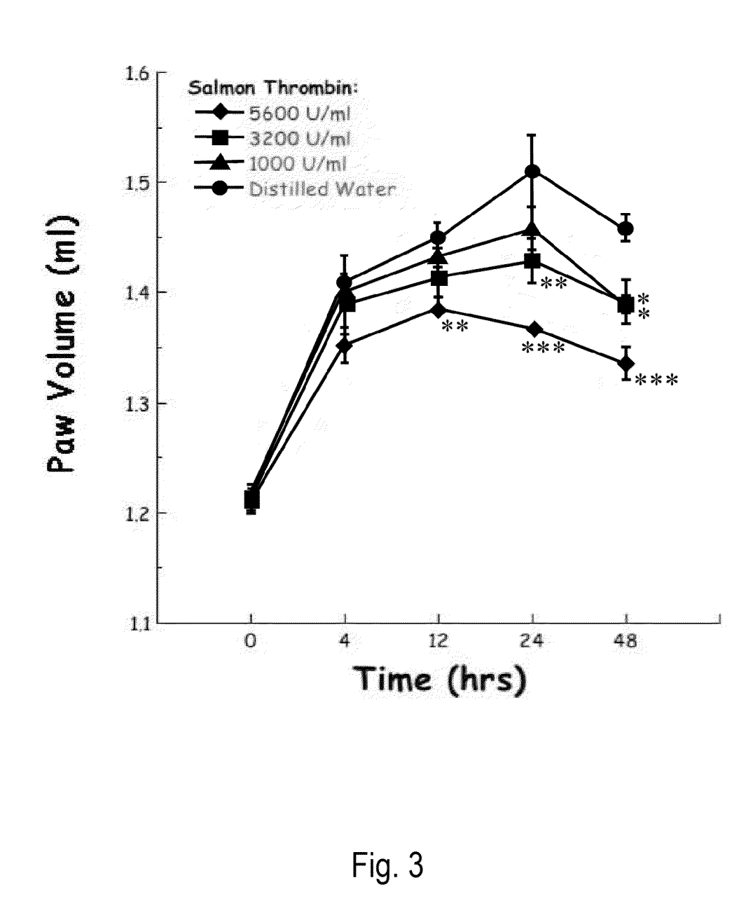

Rats receiving the salmon thrombin treatment had significantly reduced swelling and inflammation at 12, 24, and 48 hours post-surgery compared to the animals treated with distilled water (FIG 3.) All rats gained weight, and the treatment was well tolerated as assessed by behavioral observations.

Thus, the present invention provides pain relief, hemostasis, and anti-inflammation in a single stable product (dressing) suitable for field, pre-hospital, or hospital use. A one-time application of the dressing is safe, effective, and long-lasting, without the addictive properties or need for multiple applications of other therapeutic treatments.

The present invention has been described by way of example and in terms of preferred embodiments. However, it is to be understood that the present invention is not strictly limited to the particularly disclosed embodiments. To the contrary, various modifications, as well as similar arrangements, are included within the spirit and scope of the present invention. The scope of the appended claims, therefore, should be accorded the broadest possible interpretation so as to encompass all such modifications and similar arrangements.

### REFERENCES

Brennan T J, Vandermeulen E P, Gebhart G F. 1996. Characterization of a rat model of incisional pain. Pain 64:493-501.

Brennan T J. 1999, Postoperative models of nociception. ILAR Journal 40(3):1-9.

Garcia P S, Gulati A, Levy J H. 2010. The role of thrombin and protease-activated receptors in pain mechanisms. Thrombosis and Hemostasis 103(6): 1145-1151.

MacPhee M J et al. 2004. Hemostatic sandwich bandage. U.S. Pat. No. 6,762,336.

Mather L E. 2010. The acute toxicity of local anesthetics. Expert Opin Drug Toxicol. 6(11):1313-32.

Michaud S E, Wang L Z, Korde N, Bucki R, Randhawa P K, Pastore J J, Falet H, Hoffmeister K, Kuuse R, Uibo R, Herod J, Sawyer E, Janmey P A. 2002. Purification of salmon thrombin and its potential as an alternative to mammalian thrombins in fibrin sealants. Thrombosis Research. 107:245-254.

Pusateri A E, Holcomb J B, Kheirabadi B S, Alum H B, Wade C E, Ryan K L, 2006. Making sense of the preclinical literature on advanced hemostatic products J. Trauma 60(3):674-82.

Rothwell S W, Reid T J, Dorsey J, Flournoy W S, Bodo M, Janmey P A, Sawyer E, 2005. A salmon thrombin-fibrin bandage controls arterial bleeding in a swine aortotomy model. J Trauma 59(1):143-149.

Sawyer E S, et al. 2010. A method of using salmon thrombin to alleviating pain. U.S. Pat. No. 8,771,684.

Smith J R. Syre P, Oake S A, Nicholson K J, Weisshaar C L, Cruz K, Bucki R, Baumann B C, Janmey P A, Winkelstein B A, 2013. Salmon and human thrombin differentially regulate radicular pain, glial-induced inflammation, and spinal neuronal excitability through protease-activated receptor 1. PloS One 8(11):e80006.

Suo Z, Citron B A, Festoff B W. 2004. Thrombin: a potential proinflammatory mediator in neurotrauma and neurogenerative disorders. 2004. Curr Drug Targets Inflamm Allergy 3(1):105-114.

Spotnitz W D, Burks S. 2008. Hemostats, sealants, and adhesives: components of the surgical toolbox. Transfusion 48:1502-1516.

Weisshaar C L, Winer J P, Guarino B B, Janmey P A, Winkelstein B A. 2011. The potential for salmon fibrin and thrombin to mitigate pain subsequent to cervical nerve root injury. Biomaterials 9738-46.

Wu J, Yang S, Xi G, Song S, Fu G, Keep R F, Hua Y. 2008. Microglial activation and brain injury after intracerebral hemorrhage. Acta Neurochir Suppl 105:59-65.

Whiteside G T, Harrison J, Boulet J, Mark L, Gottshall S, Walker K. 2004. Pharmacological characterization of a rat model of incisional pain. British J Pharmacology 141:85-91.

Winkelstein B A, Janmey P A, Sawyer E S. 2009. Method of using salmon thrombin to alleviate central nervous system-mediated pain. U.S. patent application Ser. No. 12/582,040.

What is claimed is:

1. A wound dressing, comprising:
a backing material; and
thrombin, applied to the backing material.

2. The wound dressing of claim 1, wherein the backing material is at least partially biodegradable.

3. The wound dressing of claim 2, wherein the backing material includes biodegradable polyester.

4. The wound dressing of claim 3, wherein the backing material includes electrospun polycaprolactone.

5. The wound dressing of claim 1, wherein the backing material includes material selected from glycolic acid-based polymers, lactic acid-based polymers, glycolic acid-based copolymers, and lactic acid-based copolymers.

6. The wound dressing of claim 1, wherein the thrombin includes salmon thrombin.

7. The wound dressing of claim 1, further comprising at least one of the fibrinogen, collagen, alginates, synthetic polymer, antibacterial factors, and antiviral factors, applied to the backing material or added to the thrombin.

8. The wound dressing of claim 1, wherein the thrombin is adsorbed onto the backing material.

9. The wound dressing of claim 1, wherein the wound dressing is in a lyophilized state.

10. The wound dressing of claim 1, further comprising a detergent solution residue on the backing material.

11. A wound dressing product, comprising an airtight wrapping in which the wound dressing of claim 1 is enclosed.

12. A process of fabricating a wound dressing, comprising applying thrombin to a backing material.

13. The process of claim 12, further comprising preparing the thrombin from the plasma of a salmonid.

14. The process of claim 12, wherein the thrombin is applied to the backing material by adsorbing the thrombin onto the backing material.

15. The process of claim 14, further comprising applying a detergent solution to the backing material prior to adsorbing the thrombin onto the backing material.

16. The process of claim 15, wherein the detergent solution is 0.5 mg/ml Tween 80.

17. The process of claim 14, further comprising lyophilizing the backing material and adsorbed thrombin.

18. The process of claim 12, further comprising adding at least one of fibrinogen, collagen, alginates, synthetic polymers, antibacterial factors, and antiviral factors, to at least one of the thrombin and the backing material.

19. The process of claim 12, wherein the backing material is a biodegradable polyester.

20. A process of manufacturing a wound dressing product, comprising vacuum-sealing the wound dressing of claim 12 within an air-tight wrapping.
